(19) United States
(12) Patent Application Publication
(10) Pub. No.: US 2005/0250145 A1
(43) Pub. Date: Nov. 10, 2005

(54) On-line chemical reaction system

(76) Inventors: Atsumu Hirabayashi, Kodaira (JP); Yoshinobu Kohara, Mitaka (JP); Kazunori Okano, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/122,045

(22) Filed: May 5, 2005

(30) Foreign Application Priority Data
May 10, 2004 (JP) 2004-139305

(51) Int. Cl. C12Q 1/68

(52) U.S. Cl. 435/6

(57) ABSTRACT

In enzymatic reaction carried out batch-wise, loss of the sample cannot be ignored, and according to the conventional technologies aiming at diminishment of the loss of the sample, a long time is required for reactions. In the present invention, the reaction part in which a chemical substance is immobilized is filled with a sample solution, and the sample solution is held between air at both ends for inhibition of mixing with a buffer solution. The sample solution is provided utilizing a sample introduction part, etc.

### INCORPORATION BY REFERENCE

The present application claims priority from Japanese application JP2004-139305 filed on May 10, 2004, the content of which is hereby incorporated by reference into this application.

### BACKGROUND OF THE INVENTION

The present invention relates to a chemical reaction of a trace amount of a sample solution. Particularly, the present invention relates to a reaction of a trace amount of a biological sample, namely, proteins, peptides, lipids, sugars and DNA.

Some technologies aiming at improvement of proteolytic activity of enzymes in enzymatic reaction and reduction in loss of samples by employing on-line systems have been developed. For example, JP-A-9-313196 discloses a process of enzymatic reaction which uses chitosan beads (0.5-3 mm in diameter) on which enzymes are immobilized. According to this process, enzyme immobilized beads are added to a sample solution and the reaction is accelerated while dispersing the immobilized enzyme in the sample solution using a method such as shaking. Since enzyme is immobilized, the enzyme activity can be substantially increased, but a reaction time of 1-50 hours is required. Furthermore, JP-A-11-196897 discloses a technology relating to on-line enzymatic reaction aiming at reduction in loss of sample. In this technology, an enzyme immobilized carrier gel is packed in a column and feeding a sample solution to the column by a pump. According to this method, automating of system is possible, but it requires a reaction time of several hours. Moreover, JP-A-11-243997 discloses a probe array in which particles such as beads, on the surface of which a chemical substance is bonded, namely, probes are arrayed in a capillary, although this is different from the enzymatic reaction. In this example, a sample solution is introduced into a probe array to specifically bond the sample substance to the chemical substance, which can be optically detected. The chemical substances to be bonded can be varied depending on the particles, but information on optimization of reaction efficiency is not elucidated.

For acceleration of on-line enzymatic reaction, it is also effective to increase the surface area of the immobilized enzyme. For example, “Analytical Chemistry”, Vol. 72 (2000), p. 286-293 discloses a technology of forming 32 fine channels (50 μm in width, 250 μm in depth, 11 mm in length) on a silicon substrate and immobilizing an enzyme on the surface of the channels. Since the surface area on which the enzyme is immobilized can be increased, the enzymatic reaction can be completed in a short time. However, since the sample is introduced into the fine channels at a given flow rate, the water pressure for introducing the sample is very high, which affects the reaction efficiency. Furthermore, “Analytical Chemistry”, Vol. 74 (2002), p. 4081-4088 discloses an enzyme immobilized monolithic column where a porous monolithic column is formed in a capillary and an enzyme is immobilized on the monolithic surface. The surface area on which the enzyme is immobilized can be markedly increased, and hence the enzymatic reaction time is short and the throughput is improved. In addition, since the monolithic column is porous, the sample can be introduced under a relatively low water pressure. However, production of the monolithic column is troublesome and the production cost is high.

Hitherto, enzymatic reactions have been carried out mainly by solution reaction in batch-wise manner using a tube vessel, but loss of sample cannot be ignored in the case of batch-wise processing. Moreover, the enzyme activity may lower, and the batch-wise processing is sometimes disadvantageous for the chemical reaction of a trace amount of a biological sample.

On the other hand, in the conventional technologies aiming at the reduction of loss of sample, a reaction time of from several hours to several ten hours are required as mentioned above, and thus the reaction must take a long time. Moreover, as for the reaction process using beads, information on optimization of reaction efficiency has not been elucidated.

In order to solve these problems, there are needed chemical reaction processes and chemical reactors for performing chemical reaction treatment of a trace amount of a biological sample with a small loss of the sample.

Furthermore, in order to aim at reduction of loss of samples and perform the treatment in a short time, there are needed chemical reaction processes and chemical reactors which increase the collision rate of the molecules in chemical reaction.

### SUMMARY OF THE INVENTION

In carrying out a chemical reaction of a sample, the process of chemical reaction of the present invention is characterized by comprising a step of introducing a first liquid into a sample flow path including a reaction part containing a carrier, on the surface of which biomolecules are fixed, a step of introducing into the sample flow path a second liquid provided being separated from the first liquid with a gas layer, a step of introducing a sample into the gas layer, and a step of transferring the first solution, the sample and the second solution so that the sample transfers relatively with the carrier. In the case of carrying out a chemical reaction of a trace amount of a sample using particles having biomolecules fixed on the surface, if a buffer or the like (the above first liquid and second liquid) which is a carrier liquid contacts with the sample, there is a problem that loss of the sample caused by diffusion in the flow path cannot be ignored, while by employing the above process, the loss of the sample caused by diffusion in the flow path can be avoided. Furthermore, there is another problem that when the sample is in a trace amount, recovery of the sample lowers if the sample is lost during transportation of the sample to the reaction part. However, by employing the above process, the loss of the sample caused by transportation of the sample can be avoided.

Here, there may be a first gas layer between the first solution and the sample, and there may be a second gas layer between the second solution and the sample. Moreover, the carrier may be a plurality of fine particles, and the reaction part may be a capillary. Furthermore, the carrier may be a structure provided in the reaction part, and the reaction part may be a capillary.

Other objects, features and advantages of the invention will become apparent from the following description of the embodiments of the invention taken in conjunction with the accompanying drawings.

### BRIEF DESCRIPTION OF THE DRAWINGS

FIG. 1(a) shows a structure of the chemical reactor according to one example of the present invention.

FIG. 1(b) shows a structure of the chemical reactor according to one example of the present invention.

FIG. 1(c) shows a structure of the chemical reactor according to one example of the present invention.

FIG. 2 is a control system diagram of the chemical reactor according to one example of the present invention.

FIG. 3(a) shows a typical feeding protocol in the chemical reactor according to one example of the present invention.

FIG. 3(b) shows a typical feeding protocol in the chemical reactor according to one example of the present invention.

FIG. 3(c) shows a typical feeding protocol in the chemical reactor according to one example of the present invention.

FIG. 3(d) shows a typical feeding protocol in the chemical reactor according to one example of the present invention.

FIG. 3(e) shows a typical feeding protocol in the chemical reactor according to one example of the present invention.

FIG. 3(f) shows a typical feeding protocol in the chemical reactor according to one example of the present invention.

FIG. 3(g) shows a typical feeding protocol in the chemical reactor according to one example of the present invention.

FIG. 3(h) shows a typical feeding protocol in the chemical reactor according to one example of the present invention.

FIG. 4(a) is a diagram of the chemical reactor according to one example of the present invention (schema).

FIG. 4(b) is a diagram of the chemical reactor according to one example of the present invention (schema).

FIG. 4(c) is a diagram of the chemical reactor according to one example of the present invention (schema).

FIG. 4(d) is a diagram of the chemical reactor according to one example of the present invention (schema).

FIG. 5 is a diagram of the chemical reactor according to one example of the present invention (schema).

FIG. 6(a) is a diagram of the chemical reactor according to one example of the present invention (schema).

FIG. 6(b) is a diagram of the chemical reactor according to one example of the present invention (schema).

FIG. 6(c) is a diagram of the chemical reactor according to one example of the present invention (schema).

FIG. 6(d) is a diagram of the chemical reactor according to one example of the present invention (schema).

FIG. 6(e) is a diagram of the chemical reactor according to one example of the present invention (schema).

FIG. 6(f) is a diagram of the chemical reactor according to one example of the present invention (schema).

FIG. 7 shows an operation sequence of the chemical reactor in accordance with the feeding protocol according to one example of the present invention.

FIG. 8 shows a structure of reaction part 1 in the chemical reactor according to one example of the present invention.

FIG. 9 shows a relation between the time required for introduction of sample and the volume needed.

FIG. 10 is a schematic view of the chemical reactor according to one example of the present invention in which a structure is disposed in the flow path.

FIG. 11 shows a result of enzymatic digestion reaction of protein according to one example of the present invention.

FIG. 12 shows a diagram of analytical protocol of protein using a mass spectrometer, and a diagram of an analytical system (shotgun analytical system) in which the chemical reactor according to one example of the present invention is incorporated.

FIG. 13 shows details of the portion of 2D-HPLC.

FIG. 14 shows a diagram of protocol of structural analysis of oligosaccharide (analysis of oligosaccharide sequence) using a mass spectrometer, and a diagram of an analytical system in which the chemical reactor according to one example of the present invention is incorporated.

FIG. 15 is a diagram of analytical system in which a plurality of chemical reactors is operated in parallel.

### Description of Reference Numerals

1: Reaction part 1, 2: Valve, 3: Air introduction port, 4: The second pump, 5: Sample introduction port, 6: The first pump, 7: Thermal chamber, 8: Discharging port, 9: Buffer introduction port, 10: Capillary, 11: Glass beads, 12: Another quartz capillary, 13: Capillary, 14: Another capillary, 15: Valve, 16: Discharging port, 28: Sample introduction port, 29: Flow path, 30: Flow path, 31: Buffer discharging port, 32: Valve, 40: Silicon substrate, 41: Flow path, 42: Structure, 43: Glass substrate, 44: Hole, 47: Primary separation column, 48: Liquid reservoir, 49: Pump, 50: Mixer, 51: Valve, 52: 6-port switching valve, 53: Trap column, 54: Secondary separation column, 55: Liquid reservoir, 56: Pump, 57: Mixer, 58: Area

### DETAILED DESCRIPTION OF THE INVENTION

One constructive example of the chemical reactor is characterized by having a reaction part containing a plurality of fine particles, a first tube and a second tube connected with one end and another end of the reaction part, respectively, a sample introduction means which is connected with the first tube and introduces a sample, and a first pump and a second pump for controlling the transfer of the sample in the reaction part. Here, the sample introduction means may have at least a first flow path and a second flow path, and the disposition of the first flow path and that of the second flow path into which the sample is introduced may be exchanged by rotation, whereby the sample introduced into the second flow path may be introduced into the reaction part. The sample introduction means may have a sample holding part, and the sample introduced into the sample holding part may be forced out by gas or liquid subsequently introduced into the sample holding means, thereby to introduce the sample into the reaction part. Furthermore, a thermal chamber may be provided, and the sample introduction means and the reaction part may be disposed in the thermal chamber. Moreover, a thermal chamber may be provided, and the reaction part may be disposed in the thermal chamber.

Another constructive example of the chemical reactor is characterized by having a first flow path, a second flow path provided with a reaction part containing a plurality of fine particles, a member for exchanging the disposition of the first flow path and that of the second flow path, a first tube connected with one end of the first flow path or the second flow path, a second tube connected with another end of the first flow path or the second flow path, and a first pump connected with the first tube and a second pump connected with the second tube.

The above chemical reactor may be used alone or may be incorporated into an on-line chemical reaction system, a mass spectrometric system or the like.

The time required for introduction of sample can be reduced by using the chemical reactor of the present invention. Furthermore, the volume set as an amount to be introduced can be surely introduced, and loss of the sample can be inhibited. In addition, the efficiency of chemical reaction in the sample flow path can be enhanced by bringing about turbulent flow or transition flow of the sample in the sample flow path which contains a carrier on which a chemical substance is immobilized. Furthermore, the reaction efficiency between the chemical substance immobilized on the carrier and the sample molecules in the solution can be enhanced by increasing the collision rate of them. By enhancing the reaction efficiency in this way, the treatment can be completed in a short time and besides the biological sample in a trace amount can be subjected to a treatment of chemical reaction with small loss of the sample.

Furthermore, according to an analytical system in which the chemical reactor is incorporated, the throughput can be markedly improved by the high reaction efficiency of the chemical reactor. Moreover, since a trace amount of a sample can be treated in on-line, loss of the sample can be inhibited and the whole system can be made higher in sensitivity.

### DESCRIPTION OF PREFERRED EMBODIMENTS

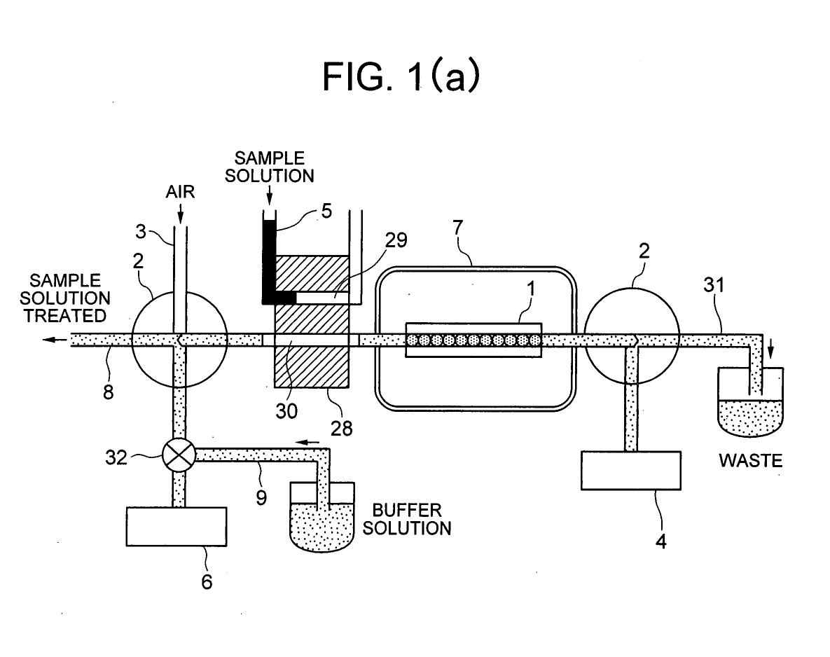

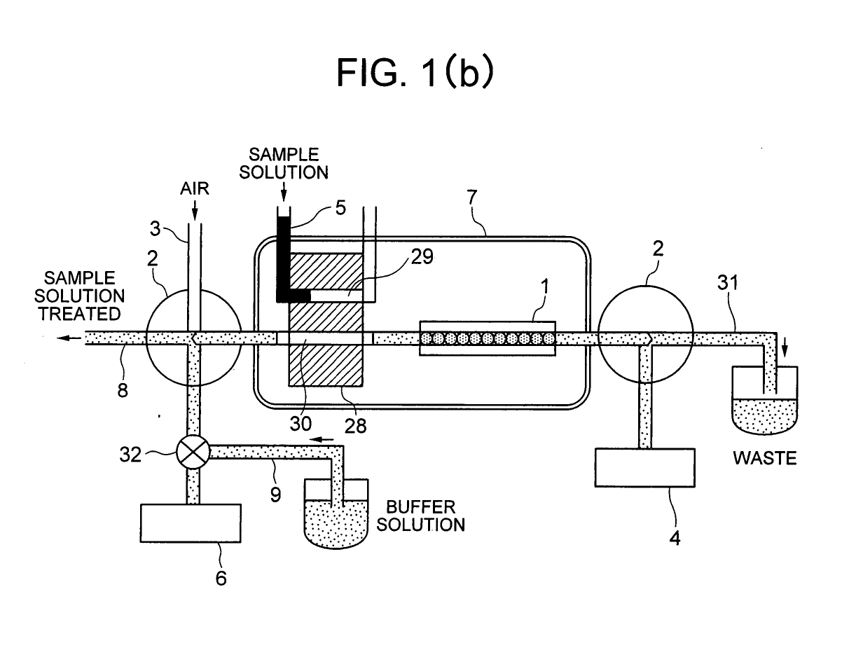

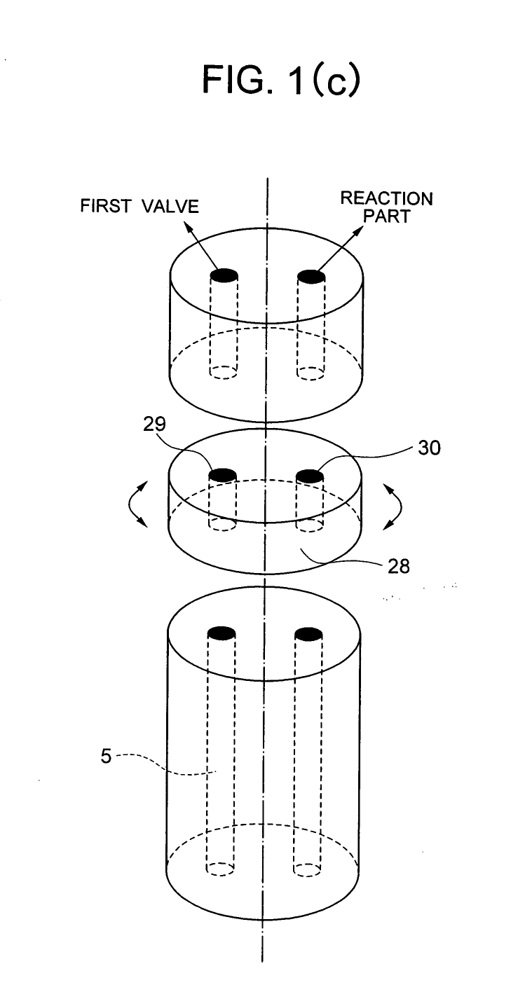

FIG. 1(a) shows a diagram (schema) of the chemical reactor according to one example of the present invention. A chemical substance is immobilized in the tubular reaction part 1. A buffer solution is filled in a valve 2, the reaction part 1 or the like. First, a given volume of air is introduced from an air introduction port 3 by a second pump 4. Next, the valve 2 is switched and air is introduced into a sample introduction part 28 by a first pump 6 and the second pump 4, and both ends of the air are in such a state as spreading over the both ends of the sample introduction part 28. A sample solution (shown by black color) is introduced into a sample introduction port 5 provided at the sample introduction part 28. In the sample introduction part 28, a flow path 29 into which the sample is introduced and a flow path 30 into which air is introduced are changed over with each other by rotation of the sample introduction part 28, or the like. As a result, both ends of the sample solution are in the state of being held between air, and the sample solution is inhibited from admixing with the buffer solution. The sample solution, the both ends of which are held between air, is introduced into the reaction part 1 by the first pump 6 and the second pump 4.

The reaction part 1 is kept at a given temperature, for example, about 37° C. by a thermal chamber 7. The sample solution is reciprocated by a given volume at a given flow rate in the reaction part 1 by the first pump 6 and the second pump 4. The solution is introduced from one side and simultaneously pressurized on another side by the pumps 4 and 6, and thus the delay of transfer of the sample solution is inhibited. By repeating the reciprocation for a given period, the chemical reaction is completed in the reaction part 1. The sample which has been subjected to the chemical reaction (reaction product) is discharged from a discharge port 8 through valve 2. Thereafter, the reaction part 1 and the valve 2 are cleaned with a buffer solution introduced from a buffer solution introduction port 9. The buffer solution used for cleaning (waste) is discharged from a buffer solution discharging port 31. During the storage without introduction of sample, the temperature of the reaction part 1 is changed to about 4° C. to inhibit change of the immobilized chemical substance. In this way, the reaction part 1 can be used repeatedly for more than 1 month.

FIG. 1(c) shows a diagram of the sample introduction part 28. The sample introduction part 28 is rotated on a dotted line as an axis by a rotating means (not shown) to change the disposition of the flow paths with each other. The sample introduction part 28 shown in FIG. 1(c) has at least two flow paths, and the sample solution (sample) is introduced into at least one flow path, which is changed over with other flow path containing gas or liquid, thereby introducing the sample solution (sample) into the reaction part 1.

In order to rapidly transfer the sample solution, the both ends of which are held between air, the inner diameter of the pipe which connects the flow path 29 into which the sample is introduced, the reaction part 1, the valve 2, and the discharging port 8 (such as a quartz capillary) is desirably 70 microns or more. This is because within this range of the inner diameter, conductance of the tube can be reduced and transfer of the sample solution held between air can be controlled at a high accuracy. Moreover, the internal volume of them is preferably about 3-5 times the volume of the sample. Within this range of the volume, the time for transfer of the sample can be shorter than the reaction time. Furthermore, the volume of air holding the sample solution therebetween is preferably in the range of 0.1-2 μL. So as not to cause mixing of the sample solution and the buffer solution, air must be in an amount of 0.1 μL or more, but if it is too large, control of transfer of the sample solution is hindered even if the inner diameter of the tube is 70 microns or more. That is, when the volume of each of air is in the range of 0.1-2 μL, transfer of the sample solution can be properly controlled.

The shorter distance between the sample introduction part 28 and the reaction part 1 is preferred from the point of speeding-up because the transfer distance of the sample, the both ends of which is held by air, is short. Therefore, as shown in FIG. 1(b), the sample introduction part 28 may be provided in the thermal chamber 7.

For the chemical reactor (the whole), it is necessary that the first pump 6, the first valve 2, the sample introduction part 28, the thermal chamber 7, the second valve 2 and the second pump 4 have power sources for driving them. Furthermore, a system control part for generically controlling the power sources is necessary. In such a system, automatic on-line treatment of a trace amount of a sample can be realized.

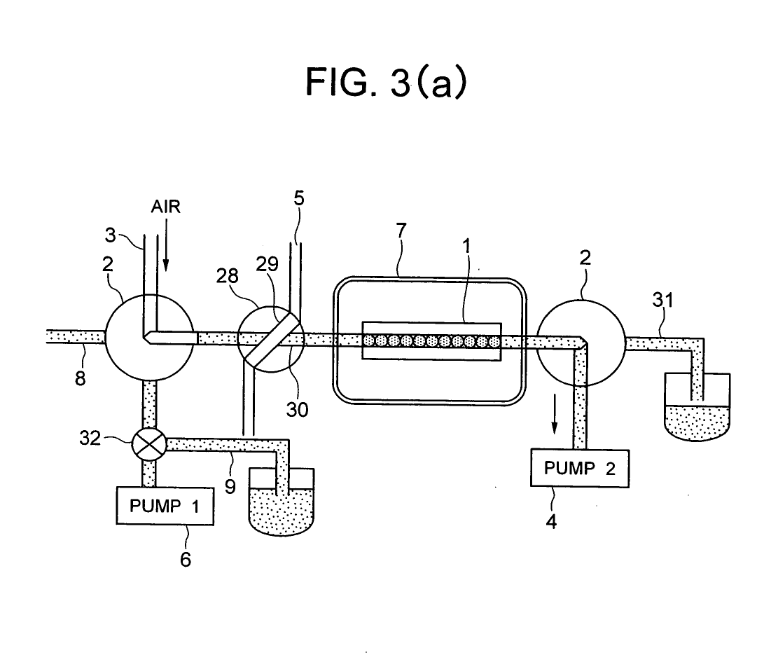

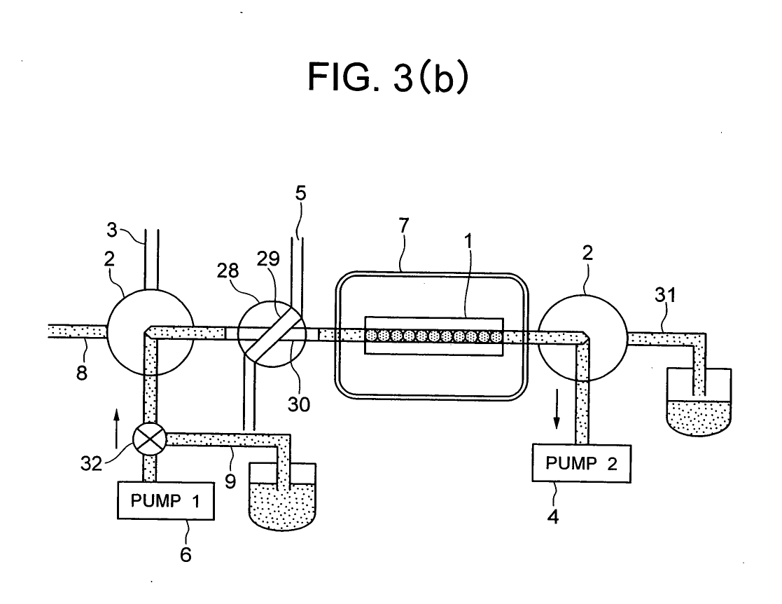

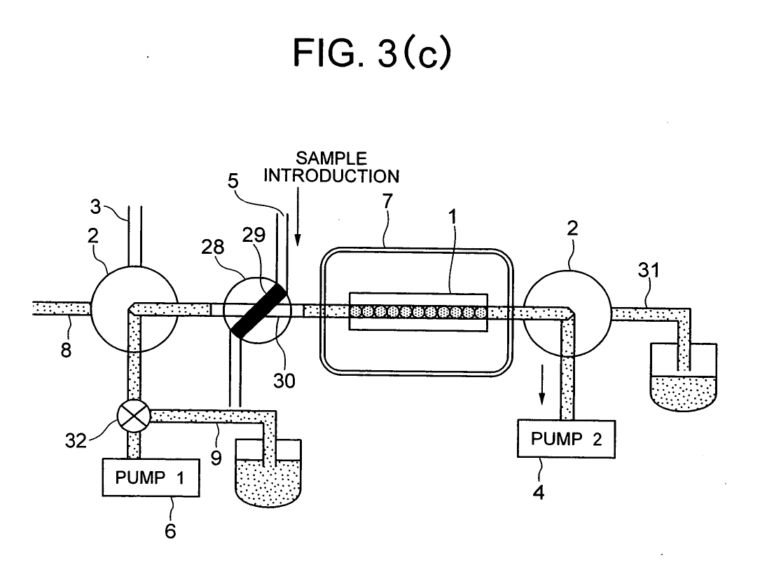

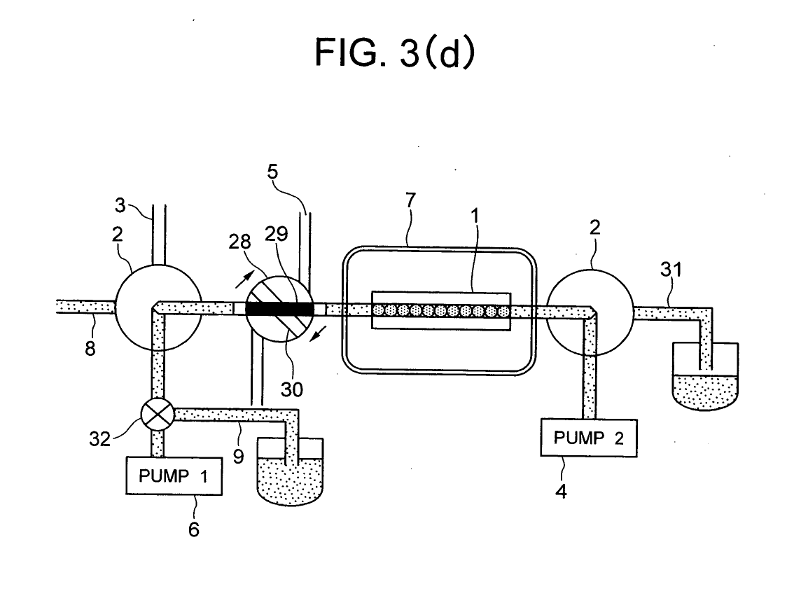

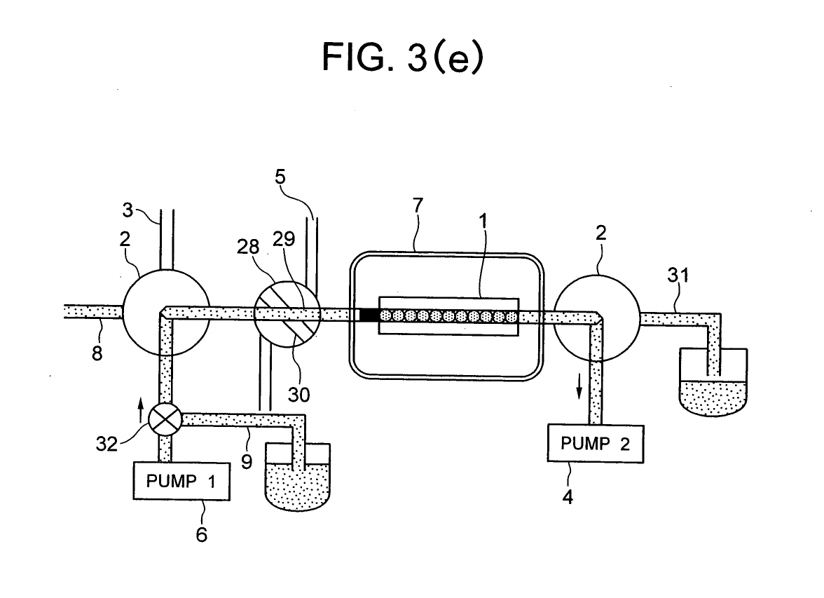

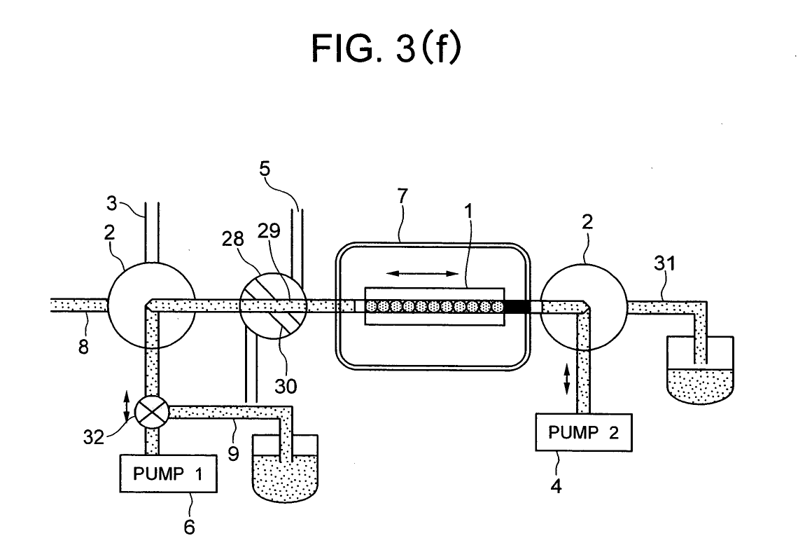

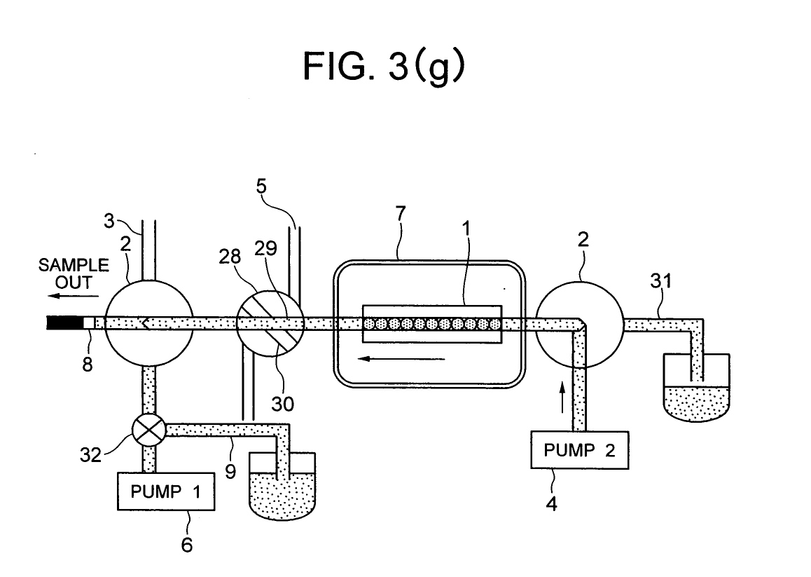

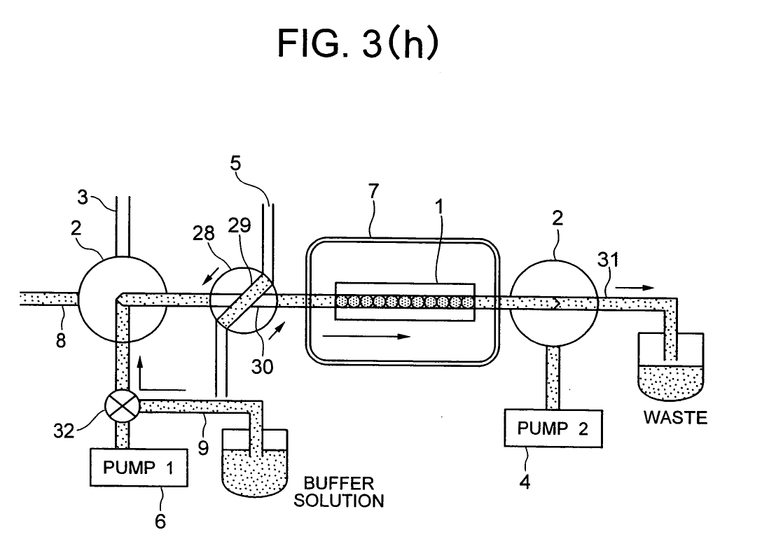

FIG. 3 diagrammatically shows a typical feeding protocol in the chemical reactor according to one example of the present invention. The internal volume of the reaction part 1 is 2 μL and the volume of sample is 5 μL. The sample can be introduced in a volume within the range of not less than 0.1 μL and not more than 100 μL in this example and other examples. The reaction part 1, the flow path 30, and the tubes (quartz capillaries of 75 μm in inner diameter) connected to pumps 4 and 6 are previously filled with a buffer solution such as Tris-HCl solution (pH 8.0) having a concentration of 10 mM. The valve 2 in this example is an inner sample loop type injector valve, but may be of other types. (a) The valve 2 filled with a buffer solution is connected with the air introduction port 3 and introduces about 7 μL of air by pump 4. Then, the valve 2 is rotated by an actuator or the like in accordance with instructions from the system control part, and the flow path is connected with the flow path going toward the pump 6.

(b) The pump 4 and pump 6 substantially simultaneously carry out introduction and pressing out at a flow rate of 5 μL/min, whereby air is introduced into the flow path 30 of the sample introduction part 28. The pumps 4 and 6 are stopped when air protrudes from both ends of the sample introduction part 28 in an amount of about 1 μL. (c) The sample (indicated by black) is introduced into the flow path 29 (having an internal volume of 5 μL) from the sample introduction port 5 by introduction or pressing out. (d) The sample introduction part 28 is rotated, whereby the flow path 29 into which the sample is introduced and the flow path 30 into which air is introduced are changed over to each other. As a result, each about 1 μL of air is disposed at both ends of the sample (about 5 μL) and the sample is inhibited from contacting or mixing with the buffer solution. (e) The sample solution held between air at both ends is introduced into the reaction part 1 at a flow rate of 5 μL/min by the first pump 6 and the second pump 4. The introduction of the sample solution is stopped when the air on the right side leaves the reaction part 1. In this state, the volume of the sample which is not introduced into the reaction part 1 is about 3 μL. The temperature of the reaction part 1 is controlled to about 37° C. by Peltier device provided in the thermal chamber 7.

(f) The second pump 4 and the first pump 6 simultaneously carry out introduction and pressing out for 0.6 minute at a flow rate of 5 μL/min so as to transfer the sample toward right side. Then, as a result of stopping the operation of the pumps 4 and 6 for about 4 seconds (waiting time), the air on the right side stops at the position of contacting with the reaction part 1. Then, the sample transfers to the left side for 0.6 minutes at a flow rate of 5 μL/min by the second pump 4 and the first pump 6, and the pumps 4 and 6 stop for about 4 seconds (waiting time). This reciprocation is repeated for a given time (the number of times), thereby accelerating the chemical reaction. In the case of enzymatic digestion reaction of protein using trypsin, the protein is converted to peptide in about 10 minutes. This setting of time can be carried out depending on the kind of the chemical substance immobilized in the reaction part and kind of the sample. (g) The valve 2 is rotated and the flow path is connected with the sample discharge port 8. The sample treated is discharged to the outside from the sample discharge port 8 at a flow rate of 5 μL/min by the second pump 4. (h) The buffer solution introduction port 9 and the flow path to the first pump 6 are connected by the valve 32 and a fresh buffer solution is introduced by the first pump 6.

Then, the valve 32 and the valve 2 are rotated to connect the flow path of the first pump 6 and that of the valve 2. The buffer solution is pressed out by the first pump 6 at a flow rate of 5 μL/min to clean the flow paths 29 and 30 in the sample introduction part and the reaction part 1. In this case, the buffer solution may be reciprocated using the first pump 6 and the second pump 4. The buffer solution used for cleaning is discharged from the discharge port 31 to the outside. Thereafter, the operation returns to the above-mentioned (a), and the next sample can be reacted. On the other hand, in case the reaction is to be terminated, the temperature of the reaction part 1 is kept at 4° C. by the thermal chamber 7 to inhibit deterioration in function of the chemical substance immobilized in the reaction part 1. The reaction part 1 can be used repeatedly, but this must be exchanged if its function deteriorates. Further, when the reaction efficiency of the reaction part is sufficiently high, the reaction completes only by passing once the sample through the reaction part 1. In this case, the reciprocation as mentioned in the above (f) is not necessarily required.

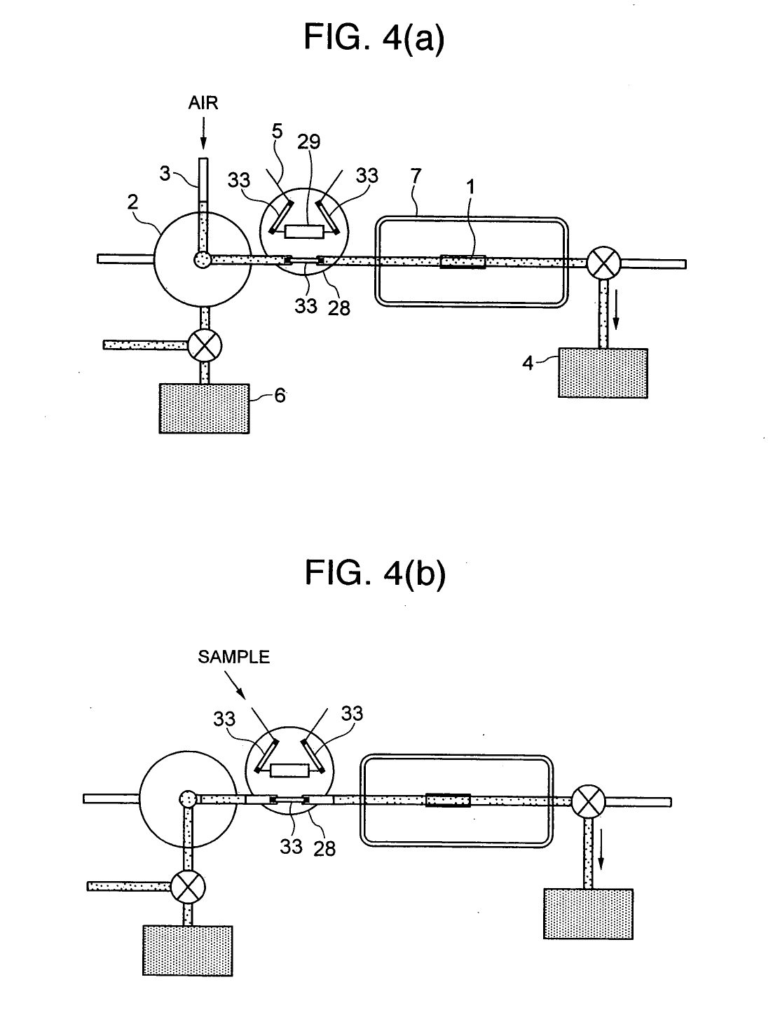

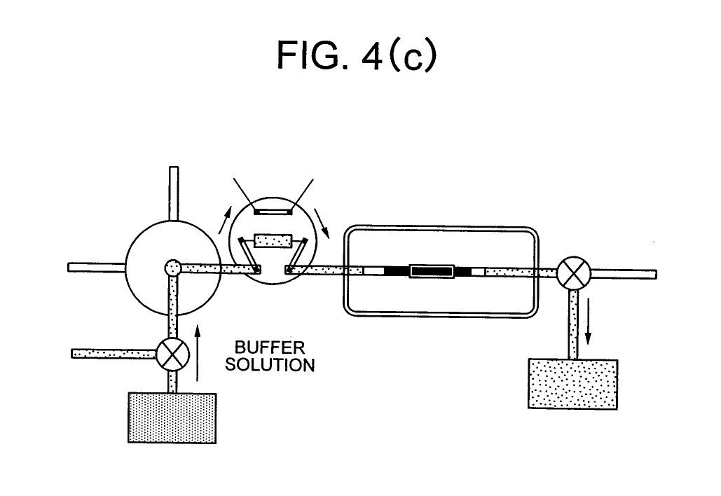

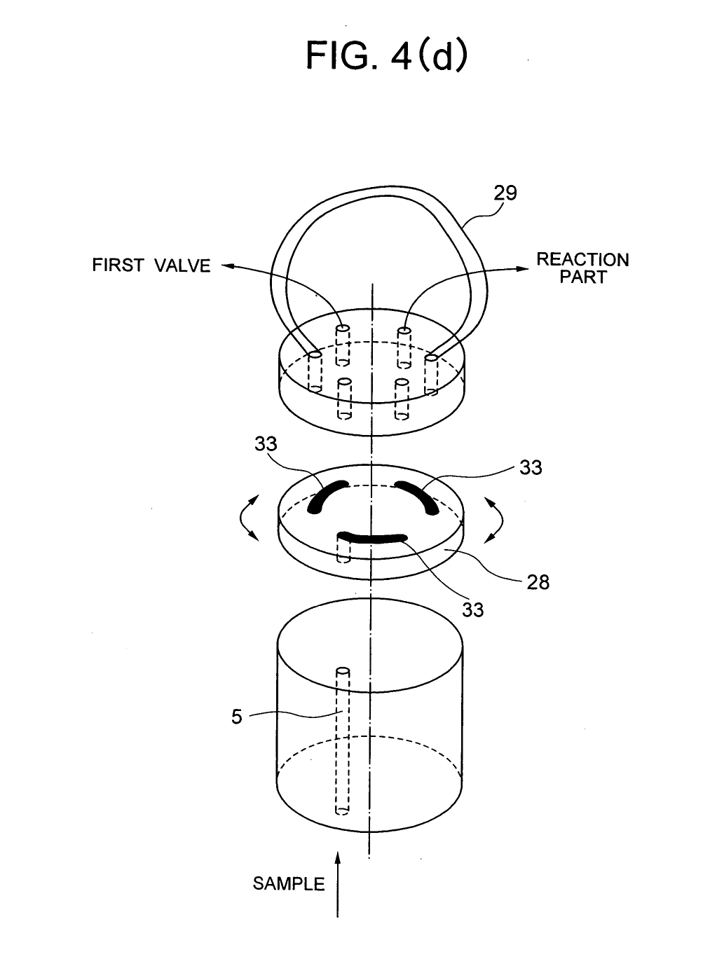

FIG. 4 shows a diagram (schema) of the chemical reactor according to one example. In this example, an outer sample loop type injector valve (6-port switching valve) is used. A diagram of the injector valve is shown in FIG. 4(d). By rotating about one-sixth the rotor part 28 where flow paths 33 are formed, the flow paths can be changed over with each other. The flow path 29 in which the sample is introduced corresponds to a sample loop having a given internal volume, and by rotating the rotor part 28 of the injector valve, the flow path 29 can be connected with valve 2 or reaction part 1.

The feeding sequence concerning with the reaction is in accordance with the explanation of FIG. 3, and the outline will be shown below. (a) The valve 2 filled with a buffer solution is connected with the air introduction port 3, from which a given volume of air is introduced by the second pump 4. Then, the valve 2 is rotated by air pressure or the like in accordance with the instructions from the system control part, and the flow path is connected with the flow path to the first pump 6. (b) The first pump 6 and the second pump 4 simultaneously carry out introduction and pressing out, respectively, at a flow rate of 5 μL/min, whereby air is introduced into the flow path 30 of the sample introduction part 28, and the pumps 4 and 6 are stopped when air protrudes in an amount of 1 μL from both ends of the flow path 33. On the other hand, the sample (indicated by black) is introduced into the flow path 29 (having an internal volume of 5 μL) from the sample introduction port 5 by introduction or pressing out. (c) The flow path 33 is rotated by one-sixth in the sample introduction part 28, whereby the flow path 29 into which the sample is introduced and the flow path 33 into which air is introduced are changed over to each other.

As a result, each about 1 μL of air is disposed at both ends of the sample (about 5 μL) and the sample is inhibited from contacting or mixing with the buffer solution. The sample solution held between air at both ends is introduced into the reaction part 1 at a flow rate of 5 μL/min by the first pump 6 and the second pump 4, and the introduction of the sample solution stops in such a state that the air on the right side leaves the reaction part 1. In this state, the volume of the sample which is not introduced into the reaction part 1 is about 3 μL. The temperature of the reaction part 1 is controlled to about 37° C. by Peltier device in the thermal chamber 7. The second pump 4 and the first pump 6 simultaneously carry out introduction and pressing out for 0.6 minute at a flow rate of 5 μL/min so as to transfer the sample toward right side. Then, as a result of stopping the operation of the pumps 4 and 6 for about 4 seconds (waiting time), the air on the right side stops at the position of contacting with the reaction part 1. Then, the sample is transferred to the left side for 0.6 minute at a flow rate of 5 μL/min by the second pump 4 and the first pump 6, and the pumps 4 and 6 stop for about 4 seconds (waiting time).

This reciprocation movement is repeated for a given time (the number of times) to accelerate the chemical reaction. Here, an injector valve having three flow paths 33 is shown, but the number of the flow paths is not limited to three. The injector valve (sample introduction part) here has a sample holding part for holding the sample solution (sample), namely, a sample loop, and the sample is introduced into the sample loop and then gas or liquid is introduced into the sample loop to discharge the previously introduced sample from the sample loop, whereby the sample is introduced into the reaction part 1. The internal volume of the sample loop corresponds to the volume of the sample, and the sample loop of about 2 μL or 5 μL in internal volume is used depending on purpose. As compared with the example shown in FIG. 3, the volume of air firstly introduced does not depend on the volume of the sample and may be about 2 μL, which is smaller than the volume in the example shown in FIG. 3, and hence the air introduction time can be shortened. This is because the internal volume of the flow path 33 rotating in the injector valve is very small and the volume of air introduced into the flow path 33 is sometimes sufficiently smaller than 1 μL.

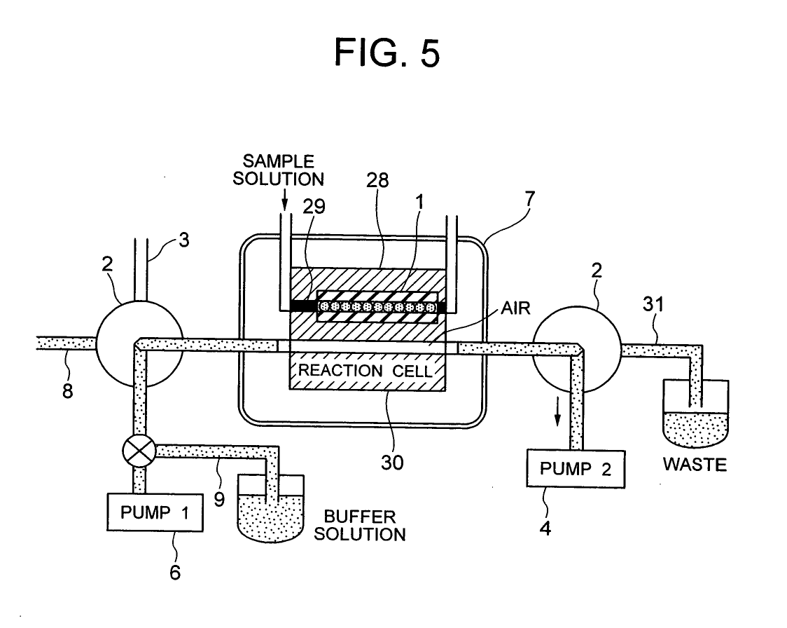

FIG. 5 shows a diagram of the chemical reactor according to one example. In this example, the flow path 29 into which the sample is introduced and the reaction part 1 are incorporated in the sample introduction part 28. In comparison with the example shown in FIG. 3, the reaction part 1 is added to the sample loop of the injector valve (6-port switching valve), and this construction is characterized in that one end of the reaction part 1 is disposed so as to contact with a switching surface of the valve. When the flow path 30 into which air is introduced, the flow path 29 into which the sample has been introduced and the reaction part 1 are changed over with each other, there can be taken such a construction that both ends of the sample are held between air. Therefore, a process of transferring the sample to the reaction part 1 becomes unnecessary, and speeding-up of the treatment is realized. Since the sample introduction part 28 is provided in the thermal chamber 7, control of the reaction temperature is easy especially when the volume of sample is great.

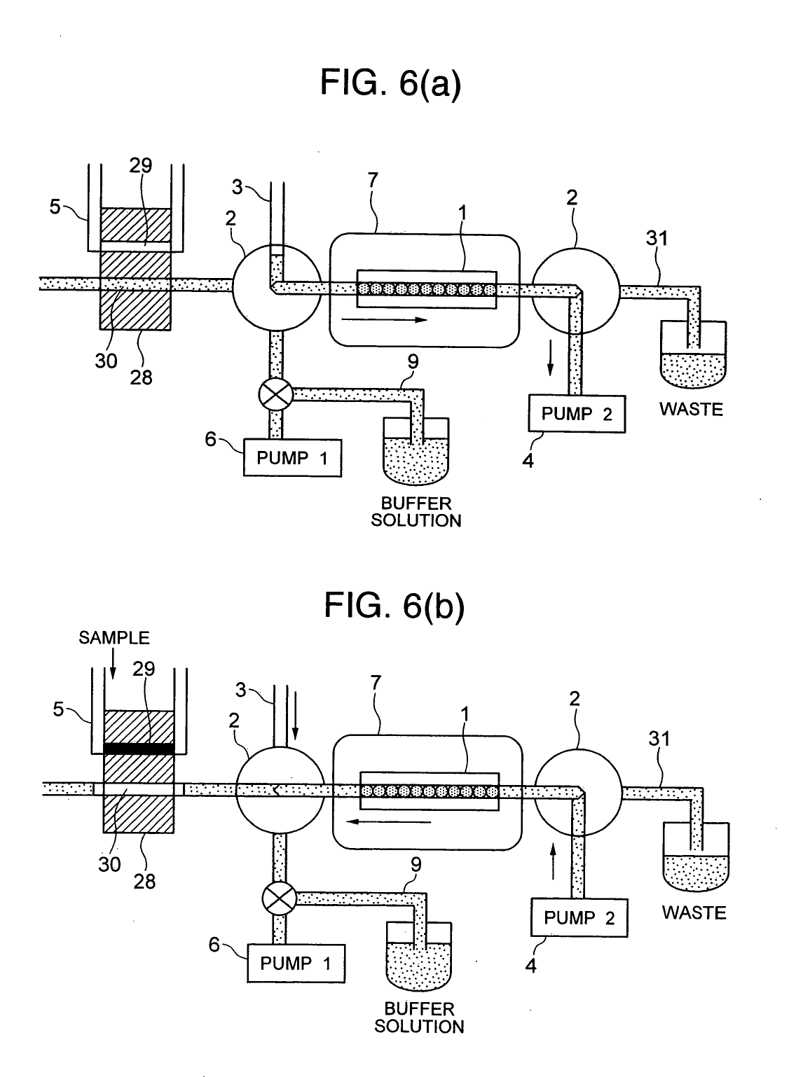

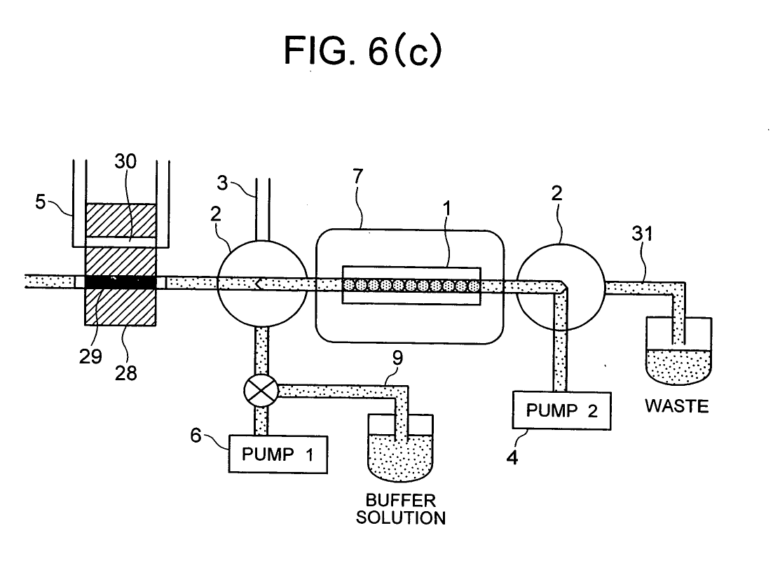

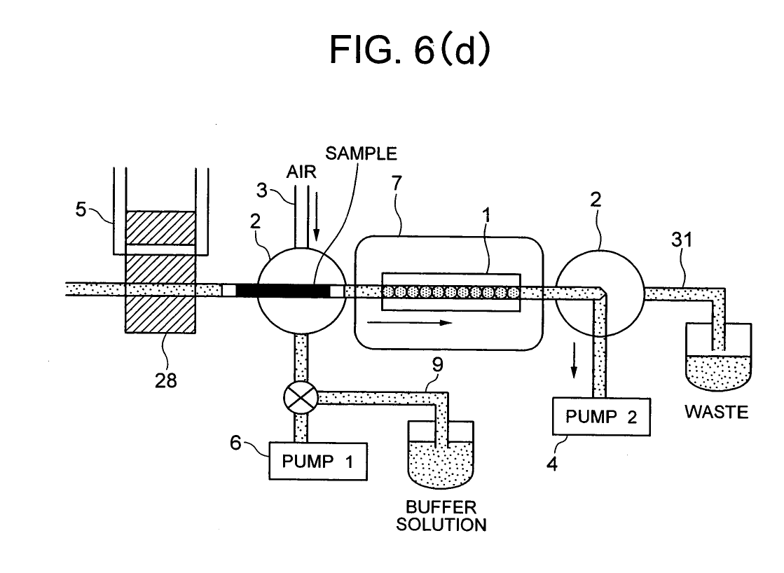

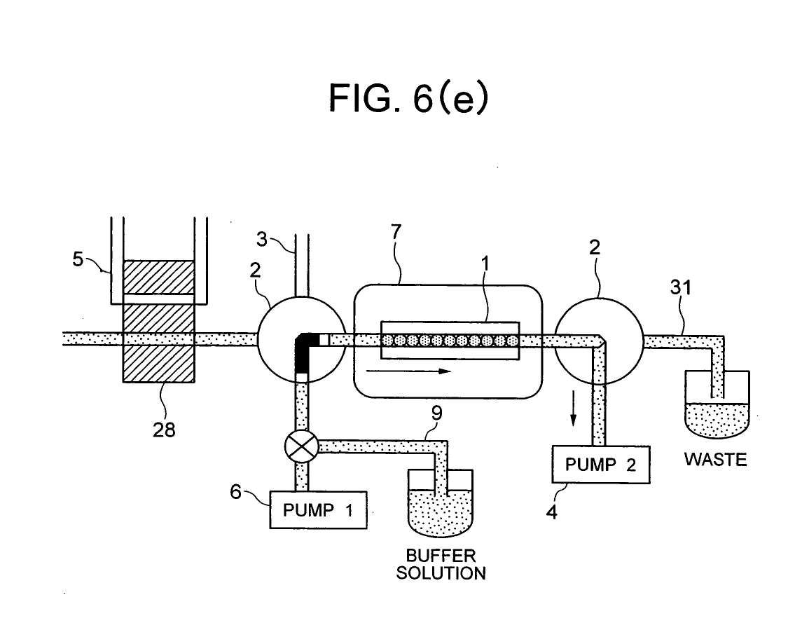

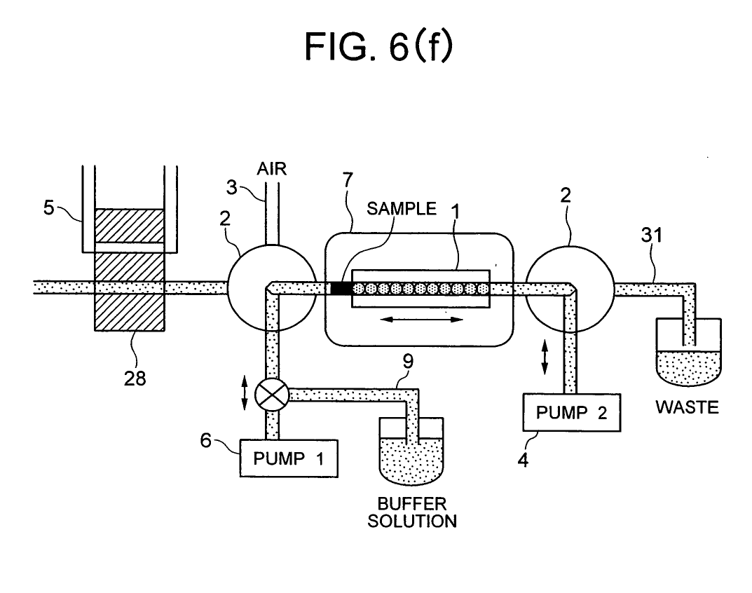

FIG. 6 shows a diagram of the chemical reactor according to one example. In this example, valve 2 is provided between the sample introduction part 28 and the reaction part 1. The feeding sequence is as follows. (a) The valve 2 filled with a buffer solution is connected with the air introduction port 3 and air in a given volume is introduced by pump 4. Then, the valve 2 is rotated, and the flow path is connected with the flow path to the sample introduction part 28. (b) The air is transferred by pressing out at a flow rate of 5 μL/min by the pump 4, but pump 4 stops in such a state that air protrudes in an amount of each about 1 μL from both ends of the flow path 29 in the introduction part 28. On the other hand, the sample (indicated by black) is introduced into the flow path 29 from the sample introduction port 5 by introduction or pressing out. (c) The sample introduction part 28 is rotated to change over the position of the flow path 29 into which the sample is introduced and the flow path 30 into which air is introduced to each other. As a result, each about 1 μL of air is disposed at both ends of the sample and thus the sample is inhibited from contacting or mixing with the buffer solution. (d) The sample held between air at both ends is introduced by the pump 4 and transferred to the reaction part 1. (e) The sample stops in such a state that the air on the right side leaves the reaction part 1. The valve 2 rotates to connect with the pump 6.

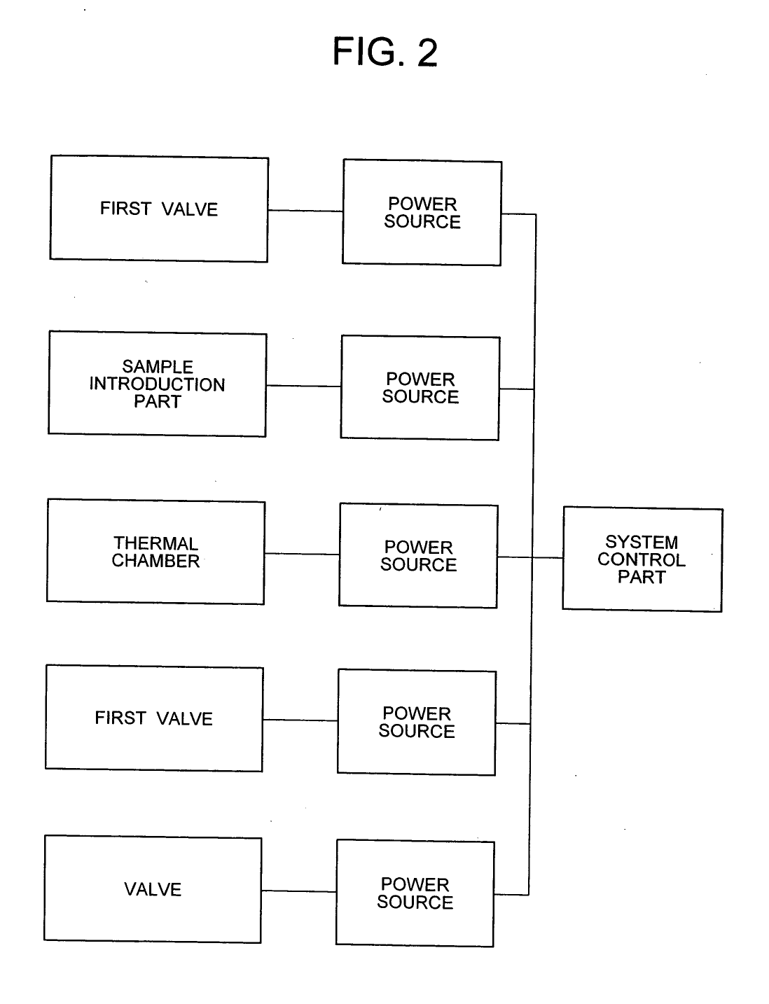

The temperature of the reaction part 1 is controlled to about 37° C. by Peltier device in the thermal chamber 7. (f) The second pump 4 and the first pump 6 simultaneously carry out introduction and pressing out for a given time at a flow rate of 5 μL/min so as to transfer the sample toward right side. Then, as a result of stopping the operation of the pumps for about 4 seconds (waiting time), the air on the right side stops at the position of contacting with the reaction part 1. Then, the sample transfers to the left side for a given time at a flow rate of 5 μL/min by the second pump 4 and the first pump 6, and the pumps 4 and 6 stop for about 4 seconds (waiting time). This reciprocation is repeated for a given time (the number of times) to accelerate the chemical reaction. The feeding protocol in the subsequent discharging of sample and cleaning is in accordance with the protocol shown in FIG. 2. In this example, the sample introduction part 28 can be disposed at a position apart from the reaction part 1, and hence the temperature of the reaction part 1 can be easily controlled.

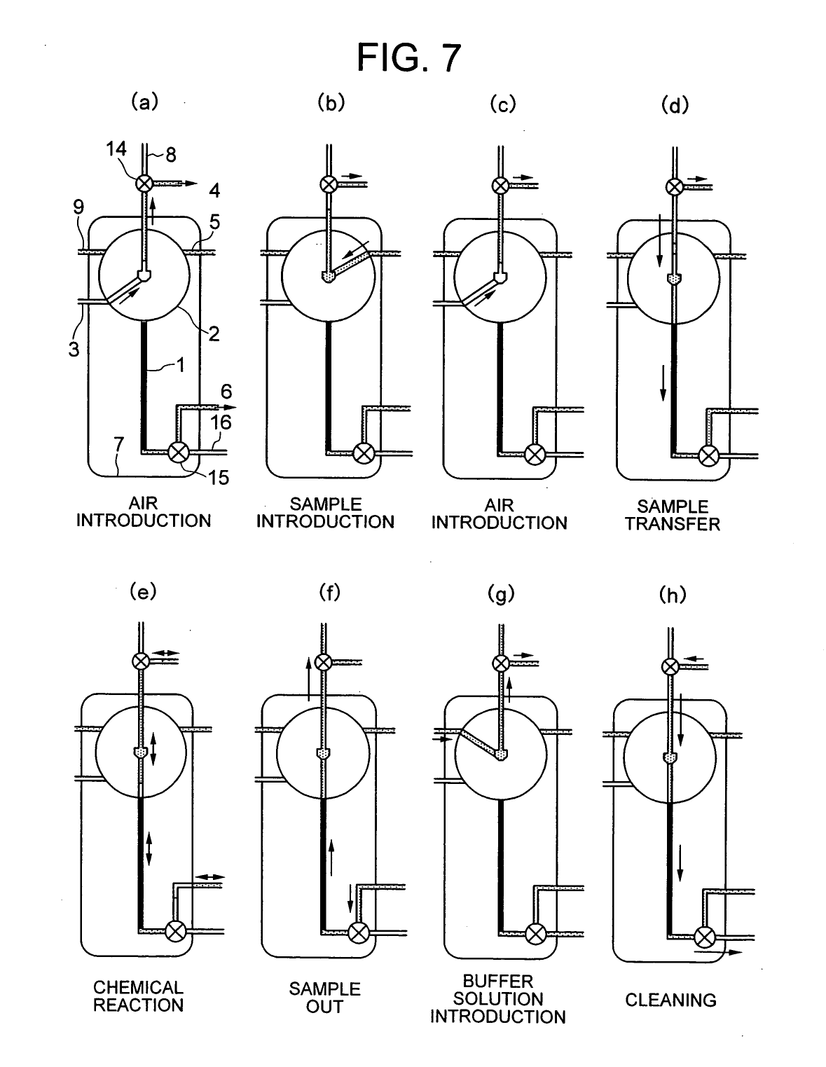

FIG. 7 shows an operation sequence of the chemical reactor in accordance with the feeding protocol according to one example of the present invention. The chemical reactor comprises reaction part 1, valve 2, air introduction port 3, sample introduction port 5, buffer introduction port 9, thermal chamber 7, first pump 6, second pump 4, and discharge port 8. Before starting of the reaction operation, the reaction part 1 is filled with a buffer solution and the piping connected with the first pump 6 and the second pump 4 is also filled with a liquid such as buffer solution. Furthermore, the temperature of the reaction part 1 is controlled to a previously given temperature by the thermal chamber 7 comprising Peltier device and the like. (a) The valve 2 connects the second pump 4 and the air introduction port 3, and a given volume of air is introduced by the second pump 4 from the valve 4 toward the piping connected with the second pump. (b) Then, the valve 2 is rotated to connect the sample introduction port 5 and the second pump 4, and a given volume of the sample is introduced by the second pump 4 from the valve 2 toward the piping connected with the second pump 4. (c) The valve 2 is again rotated to connect the second pump 4 and the air introduction port 3, and a given volume of air is introduced by the second pump 4 from the valve 2 toward the piping connected with the second pump 4.

This corresponds to the state of FIG. 3(c). (d) The valve 2 is rotated and air/sample/air introduced from the valve 2 toward the piping connected with the second pump 4 is connected to the inlet of the reaction part 1. The sample is transferred toward the first pump 6 by the first pump 6 and the second pump 4 until it fills the reaction part 1. (e) The sample reciprocates for a given time in the reaction part 1. In the case of enzymatic digestion reaction of protein using trypsin, the protein is converted to peptide in about 10 minutes. This setting of time can be carried out depending on the kind of the chemical substance immobilized in the reaction part and kind of the sample. (f) The valve 2 is connected with the discharge port 8 by the valve 14. The sample is discharged from the reaction part 1 by the first pump 6 and transferred toward the discharge port 8 and discharged to the outside. (g) The valve 14 operates to connect the valve 2 and the piping to the second pump 4. Then, the valve 2 is rotated to connect the buffer introduction port 9 and the piping going to the second pump 4, and a fresh buffer solution is introduced from the buffer introduction port 9. (h) The valve 2 is rotated and the piping connected with the second pump 4 is connected with the reaction part 1, and a fresh buffer solution is introduced into the reaction part 1. The fresh buffer solution cleans the reaction part 1 or the like by reciprocation in the reaction part 1.

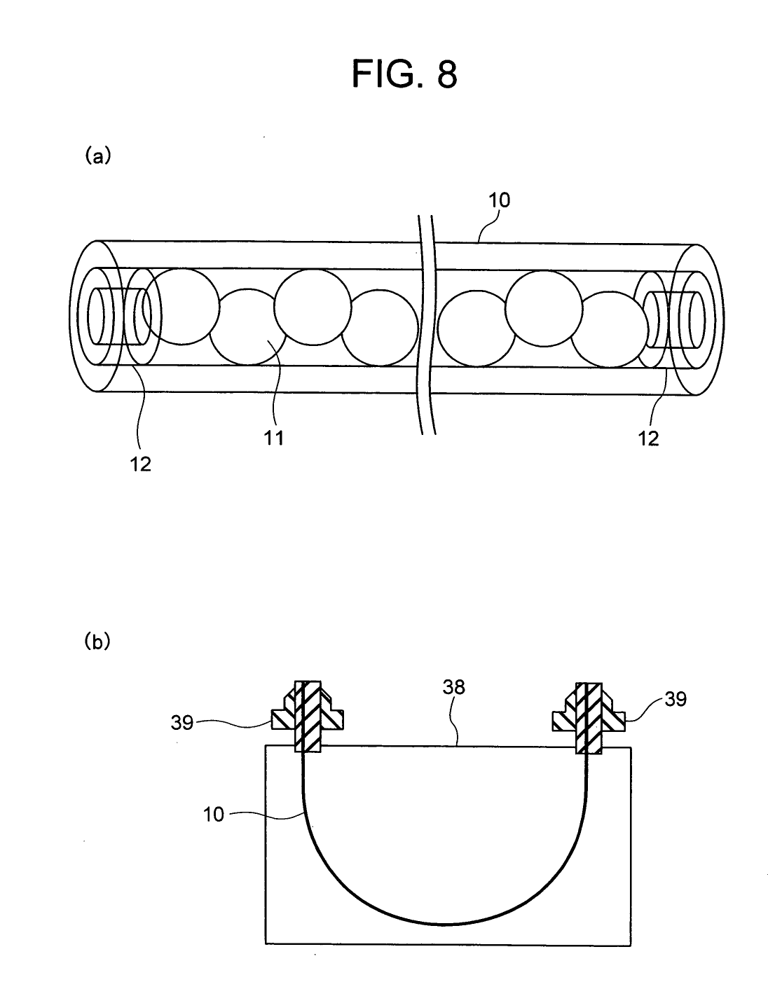

Thereafter, valve 15 is operated to connect the discharge port 16 with the reaction part 1, and the buffer solution is discharged from the discharge port 16 by the second pump 4. When the reaction process is successively carried out, the operation returns to the process of the air introduction. On the other hand, when the reaction is to be terminated, the temperature of the reaction part 1 is lowered to 4° C. by the thermal chamber 7 to inhibit the reaction part 1 from deterioration of function. The reaction part 1 can be used repeatedly, but if the function deteriorates, it must be exchanged. As shown in FIG. 8(b), when capillary 10 of the cell is fixed in a container 38 made of a material of high thermal conductivity such as aluminum and both ends of the capillary 10 can be fitted with fitting 39, exchange of the reaction part 1 can be easily performed. Here, the valve 2 is provided inside the thermal chamber, but the valve may be provided outside the thermal chamber. The sample introduction part which is omitted in FIG. 7 may be provided outside the thermal chamber as in FIG. 1(a), FIG. 3, FIG. 4 and FIG. 6, and may be provided inside the thermal chamber as in FIG. 1(b) and FIG. 5. In this example, in order to introduce the sample from the sample introduction port 5, it is preferred to supply the sample by a container such as a tube. In this case, when the inner diameter of the flow path of the valve 2 is sufficiently large, in introduction of air or sample at (a), (b) and (c), the liquid can be transferred at a high accuracy even if the flow rate of introduction is set at high flow rate of about 5 μL. Therefore, the treatment can be realized in a time similar to the time in the example shown in FIG. 1.

FIG. 8(a) shows the structure of the reaction part 1 in the chemical reactor according to one example of the present invention. In a capillary 10 of 200 mm in length (150 μm in inner diameter and 360 μm in outer diameter) are introduced about 2800 glass beads 11 (103 μm in diameter) on which trypsin is immobilized. In both ends of the capillary 10 is inserted another quartz capillary 12 (50 μm in inner diameter, 150 μm in outer diameter and 5 mm in length) for fixing the glass beads 11. It is effective to provide a coating on the inner surface of the capillaries 10 and 12 for inhibiting adsorption of the sample, but the same chemical substance as of the glass beads may be immobilized. The reaction part of such structure has a volume of the reaction part of about 2 μL.

As one example, a method of preparation of trypsin-immobilized glass beads will be explained. Immobilization of trypsin on the glass beads 11, the surface of which is modified with amino groups, can be carried out in the following manner.

1. <Substitution of carboxyl groups for amino groups on the surface of the beads> Amino group-modified glass beads (100 mg) are put in a polypropylene tube (2 mL container), and thereto is added 500 μL of a succinic anhydride solution (solvent: 1-methyl-2-pyrrolidone) having a concentration of 480 mM.

2. The succinic anhydride solution and the beads as contained in the tube are stirred at 50° C. for 60 minutes.

3. A 0.1 M boric acid buffer (pH 8.0) in an amount of 500 μL is charged in the tube, and the tube is left to stand at 20° C. for 10 minutes.

4. The beads in the tube are washed with 1 mL of pure water. This washing process is repeated six times.

5. <Activation of carboxyl groups> The beads are washed once with a mixed solution (1 mL, solvent: 0.1 M boric acid buffer (pH 6.2)) comprising 20 mM of N-hydroxysuccinimide and 0.1 M of N-ethyl-N′-3-dimethylaminopropylcarbodiimide.

6. To the beads is added a mixed solution (1 mL, solvent: 0.1 M boric acid buffer (pH 6.2)) comprising 20 mM of N-hydroxysuccinimide and 0.1 M of N-ethyl-N-3-dimethylaminopropylcarbodiimide. The beads as contained in the tube are left to stand on ice for 30 minutes (with occasional stirring), and only the beads are recovered.

7. The beads are washed with 200 μL of 0.1 M boric acid buffer (pH 6.2).

8. <Immobilization of trypsin>40 mg of trypsin is dissolved in 800 μL of 0.1 M boric acid buffer (pH 6.2), and the solution is added to the beads. The beads are left to stand at 4° C. for a whole day and night (16 hours).

9. The beads are washed with 2 mL of a 10 mM Tris-HCl solution (pH 8.0). This washing process is repeated 6 times.

10. The beads are dipped in a 10 mM Tris-HCl solution (pH 8.0) and stored at 4° C.

The above-mentioned method for immobilization of a chemical substance is not limited to immobilization of trypsin. The resulting enzyme-immobilized glass beads 11 can be packed in capillary 10 as shown in FIG. 8 by introducing the beads together with a buffer solution into the capillary 10 using a pump or the like. For observing the state of packing of the beads, the capillary 10 is preferably substantially transparent. The enzyme-immobilized glass beads 11 tend to decrease in enzymatic activity upon drying. Therefore, it is desirable that the once prepared reaction part 1 is filled with a buffer solution and covered with a lid to inhibit it from drying and is stored at 4° C. In this way, even if the reaction part 1 is repeatedly used, the enzymatic activity of the reaction part 1 can be maintained. Furthermore, as shown in FIG. 8(b), when capillary 10 of the cell is fixed in a container 38 made of a material of high thermal conductivity such as aluminum and both ends of the capillary 10 can be fitted with fitting 39, exchange of the reaction part 1 can be easily performed.

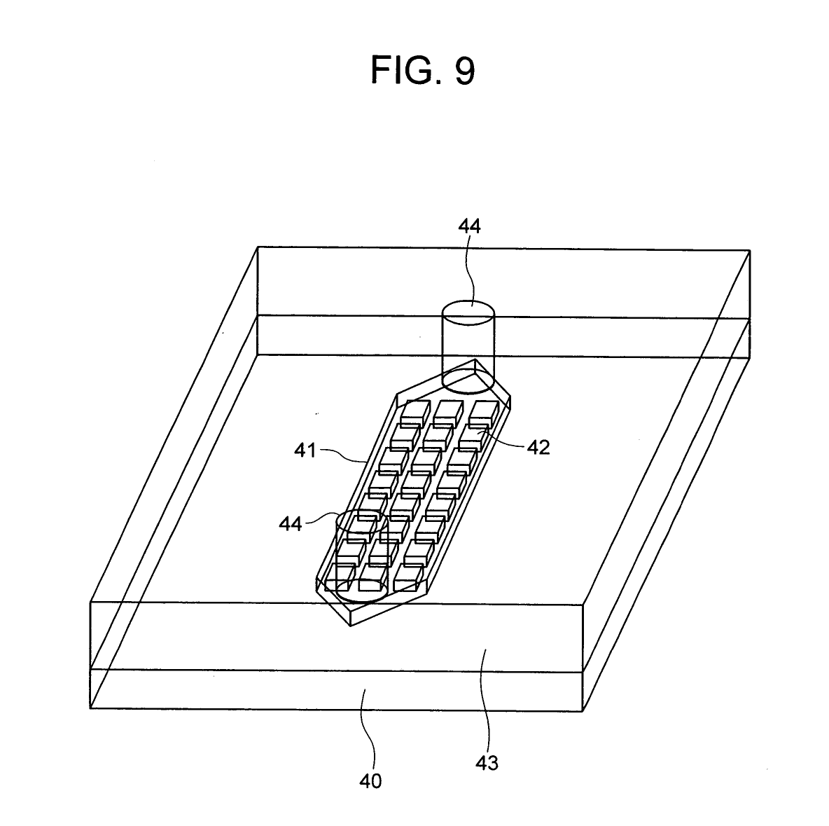

FIG. 8 shows an example where an enzyme is immobilized on glass beads and the glass beads are arranged in a line, but hard fine particles (or structure) on which an enzyme is immobilized may be disposed in the flow path, and the operation may be carried out under the above conditions. For example, as shown in FIG. 9, a flow path 41 and a structure 42 for producing turbulent flow may be provided in a silicon substrate 40 to form a cell. Holes 44 are made through a glass substrate 43 and the glass substrate is bonded to the silicon substrate 40, whereby a cell can be produced. As mentioned hereinafter, the fine particles (or structure) for forming turbulent flow are desirably hard like a glass and cannot be soft like a gel, and the fine particles may be made of a resin such as PDMS (polydimethylsiloxane).

The feeding conditions for sample in the reaction part 1 greatly relate to the efficiency of chemical reaction. For example, when the flow rate (flow velocity) is sufficiently low, the flow of the liquid is laminar flow. In this case, a movement component perpendicular to the flow of sample molecules is formed by thermal diffusion, and this thermal diffusion governs the collision against the wall surface on which the chemical substance is immobilized. In the course of this collision, the chemical reaction proceeds at a specific probability. In the case of the sample molecules being protein, the diffusion rate is about 10 μm/sec and only the sample molecules in the vicinity of the wall surface causes a chemical reaction, but most of the sample molecules present at the central portion of the flow require much time to transfer to the wall surface. That is, the chemical reaction of the whole sample molecules is difficult to take place without taking a sufficient time. On the other hand, when the flow rate (flow velocity) is sufficiently high, the flow of the liquid is turbulent flow. In this case, since turbulent diffusion fills a substantial role to improve the reaction efficiency, all of the sample molecules are apt to collide against the wall surface and the total chemical reaction efficiency is improved. Even when the flow is not a complete turbulent flow, if it is a transition flow which produces partial turbulent flow, the chemical reaction efficiency is improved as compared with the laminar flow, and thus the transition flow is advantageous.

It is generally known that when resistance coefficient C for a round tube is proportioned to Re⁻¹ of Reynolds number Re, a laminar flow is formed. On the other hand, in the case of turbulent flow, the resistance coefficient C is proportioned to Re⁰ of Reynolds number Re, and shows such an intermediate dependence that it is proportioned to about Re^{-1/2} of the Reynolds number Re in the case of transition flow which partially produces turbulent flow. Since the turbulent diffusion is effective in the case of transition flow and turbulent flow, the resistance coefficient C is proportioned to Re⁰ to Re⁻¹ of Reynolds number Re. The resistance coefficient C is proportioned to Q-2 of flow rate Q and to ΔP¹ of back pressure ΔP. Furthermore, Reynolds number Re is proportioned to the flow rate Q. Thus, it can be concluded that the turbulent diffusion is effective under the following conditions.
ΔP∝Q¹⁻² (1)

In the above formula, the case where ΔP is proportioned to Q corresponds to laminar flow and the case where ΔP is proportioned to Q² corresponds to complete turbulent flow. Actually, in many cases, it is physically difficult to realize complete turbulent flow, and the turbulent diffusion is effective unless it is laminar flow. That is, a sufficient effect can be obtained under the conditions of ΔP being proportioned to Q^{(1.5±0.4)}.

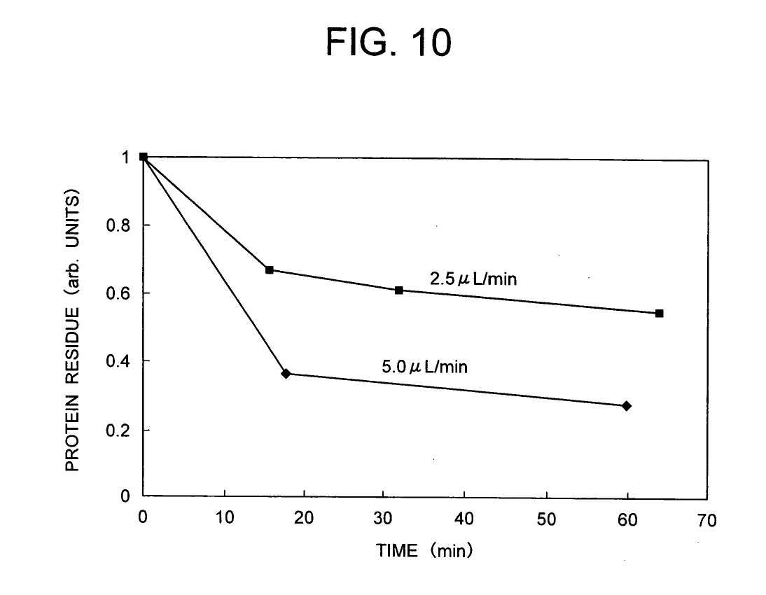

Actually, it is realistic to previously set the feeding conditions (flow rate). For example, when a pump for liquid chromatograph is used, the relation of liquid flow rate Q and back pressure ΔP for the reaction part 1 can be investigated. If from the relation, a suitable flow rate satisfying the nonlinear relation as of above formula is determined, a chemical reaction using the turbulent diffusion can be realized. FIG. 10 shows the results of enzymatic digestion reaction. The sample used is cytochrome C protein, and trypsin enzyme is immobilized in the reaction part 1. It is confirmed that when the flow rate is 5 μL/min, the back pressure ΔP is proportioned to about Q^{1.5}, which satisfies the above conditions. In FIG. 10, the ordinate axis shows the protein residue (relative value) and the abscissa axis shows the reaction time. In the case of laminar flow, the reaction is ought not to depend on the flow rate, but ought to depend on the reaction time. FIG. 10 shows that when the case of the flow rate being 5 μL/min is compared with the case of the flow rate being 2.5 μL/min, if the flow rate decreases to ½, the reaction efficiency lowers although the reaction time is the same. This is a characteristic of turbulent flow and transition flow. If the reaction is carried out at the higher liquid flow rate, the reaction efficiency is expected to be further improved, but the back pressure ΔP also conspicuously increases. Therefore, it is necessary to take care that leakage of liquid does not occur at joints of piping and capillaries.

There may be caused the problems that if air enters in the reaction part many times in the case of reciprocating the sample solution held between air at both ends in the reaction part, fine bubbles incorporate into the solution and besides the sample solution is diluted with a buffer solution. In this case, loss of the sample may be caused. Therefore, when the sample solution is reciprocated in the reaction part, it is necessary to inhibit the air present at both ends of the sample solution from contacting with the area where the chemical substance is immobilized inside the reaction part. Therefore, the sample solution firstly introduced must be in a volume as set. However, in the introduction of the sample, when the liquid held between air is introduced through the capillary as shown in FIG. 7, sometimes a given amount of the liquid is not introduced into the capillary owing to the viscosity of the liquid and change in volume of the air layer.

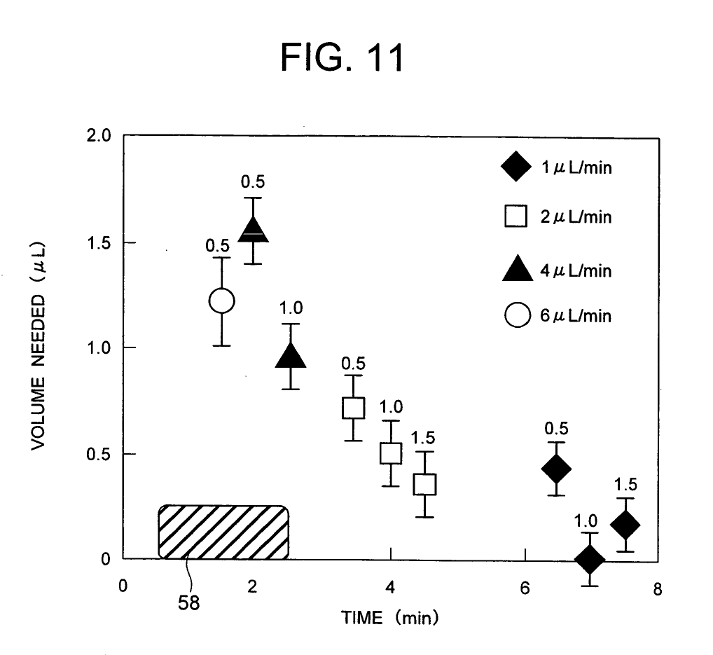

FIG. 11 shows the results of measurement of the volume needed of the sample when 1 μL of air was introduced using a quartz capillary of 150 μm in inner diameter and then 5 μL of a sample solution was introduced. This data was obtained by connecting the capillary filled with a buffer solution with a syringe filled with a buffer solution and introducing the sample solution contained in a tube by a syringe pump capable of programming. Since an actual protein solution is sometimes high in viscosity, an aqueous polyethylene glycol solution (molecular weight 1,000,000, 50 g/L) was used as the sample solution. The time required in FIG. 11 is a sum of the time for introduction of air, the time for introduction of the sample solution and the waiting time (the numerals in FIG. 11 (min)). Actually, the waiting time means a certain time for which the introduction is stopped after introduction of air, and unless the waiting time is set, the amount of sample introduced may be insufficient. Therefore, the waiting time for which the introduction is stopped was changed with respect to the flow rate during introduction, and the volume needed of the finally introduced sample was measured.

According to the results of measurement shown in FIG. 11, the amount needed of the sample introduced tends to decrease with the flow rate in introduction being lower and the waiting time being longer. The results show that when the volume needed of the sample solution introduced is to be less than 5% (less than 0.25 μL), the time required is 7 minutes or more. Since the reaction time is about 10 minutes, the time required is preferably shorter for speeding-up of the treatment.

On the other hand, according to the method of introducing a sample using the sample introduction part as shown in FIG. 3 to FIG. 6, the sample can be transferred at a speed of as high as 5 μL/min with causing substantially no need in the amount of the sample introduced. If air is previously introduced, in the case of the amount of sample being 5 μl, the time required for the introduction can be shortened to about 1 minute (area 58 in FIG. 11), and a quantitative and high-speed treatment becomes possible. When the sample introduction time may not be considered, the flow velocity of introduction may be set at low velocity.

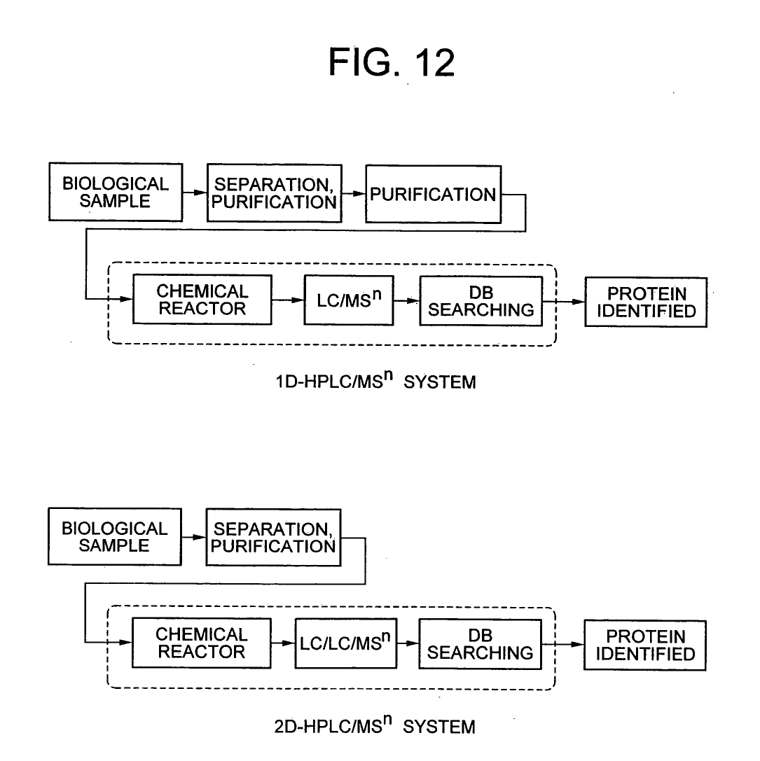

FIG. 12 shows a relation between an analytical protocol of protein using a liquid chromatograph/mass spectrometer (LC/MS) and a diagram of an analytical system in which the chemical reactor according to one example of the present invention (shotgun analytical system) is incorporated. The portion enclosed with a dotted line is the analytical system in which the chemical reactor according to one example of the present invention is incorporated. The biological sample obtained from a living organism is separated and purified by a liquid chromatograph or an affinity column. The sample (protein mixture) obtained by separation and purification is converted to peptide with an immobilized digestive enzyme such as trypsin in the chemical reactor. The peptide mixture is separated by a liquid chromatograph with reverse-phase column (1D-HPLC) or a liquid chromatograph with ion exchange column and reverse-phase column (2D-HPLC), and the separated peptide is subjected to tandem mass analysis (MSⁿ) by a mass spectrometer. The results of the mass spectrometric analysis are sent to an information processing apparatus and is subjected to database searching.

From the results of searching, the protein which is originally present is identified. The part of the chemical reactor requires 8-16 hours according to conventional batch treatment. The 2D-HPLC requires a half day and 1D-HPLC requires about 1 hour, and conventionally it requires at least 2 days including the chemical reaction stage. However, according to the chemical reactor of the present invention, the results can be obtained in about a half day, and the throughput is markedly improved. The amount of the biological sample obtained from a living organism is preferably as small as possible, and hence the loss of the sample caused by the batch treatment is a problem. If the sample is diluted, the surface area of the sample solution increases and therefore the loss due to adsorption to containers or the like cannot be ignored. According to the chemical reactor of the present invention, since the sample in a trace amount is not diluted as far as possible and on-line treatment can be carried out, the loss of the sample can be inhibited and the whole system can be enhanced in sensitivity. The database used in this analytical system may be one which has been previously constructed by input operation or one which is enhanced in version upon accessing renewed data through servers utilizing external database.

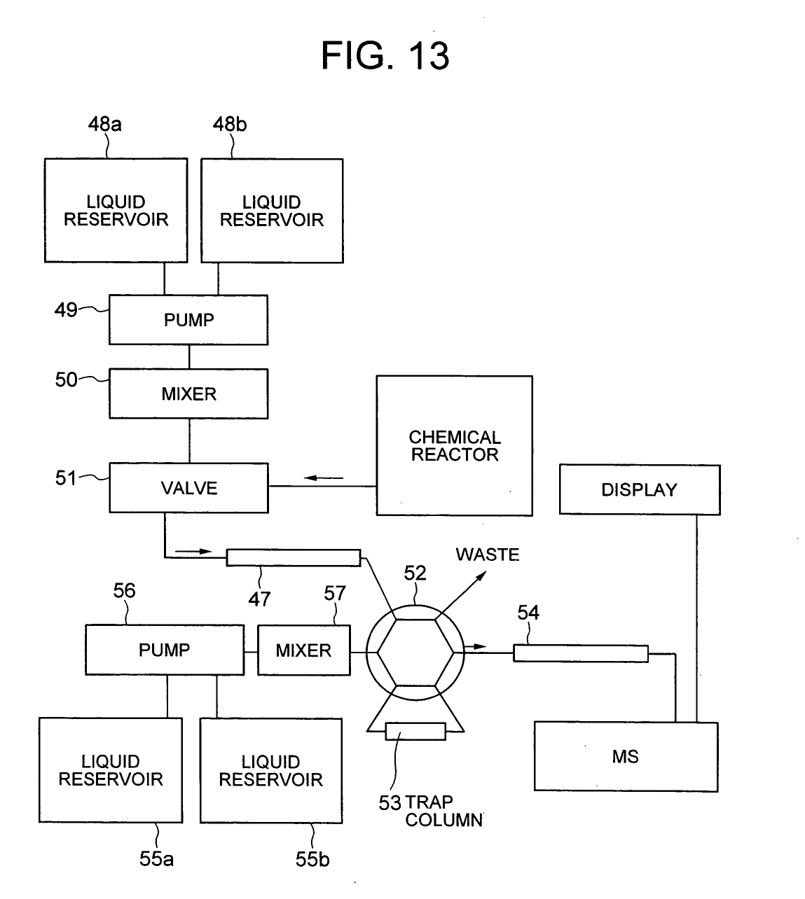

FIG. 13 shows details of the part of 2D-HPLC. It is shown that the discharge port 8 of the chemical reactor is connected on-line with an injector valve of 2D-HPLC or a trap column. Into the primary separation column 47 (ion exchange column or the like) are introduced solvents differing in composition from liquid reservoirs 48 through valve 51 by pump 49 and mixer 50. The sample separated by the primary separation column 47 is once adsorbed to trap column 53 connected with 6-port switching valve 52. Then, into the secondary separation column 54 (reverse-phase column or the like) are introduced solvents differing in composition from liquid reservoirs 55 through valve 52 by pump 56 and mixer 57. The sample separated by the secondary separation column 54 is introduced into the mass spectrometer (MS) and is subjected to mass separation. The output of MS is displayed in the display. When 1D-HPLC is used, similarly the chemical reactor is connected on-line. It is desired that in such a system the treating time in the chemical reactor is also substantially the same as the actual reaction time, and a method of introducing the sample using the sample introduction parts as shown in FIG. 3 to FIG. 6 is effective.

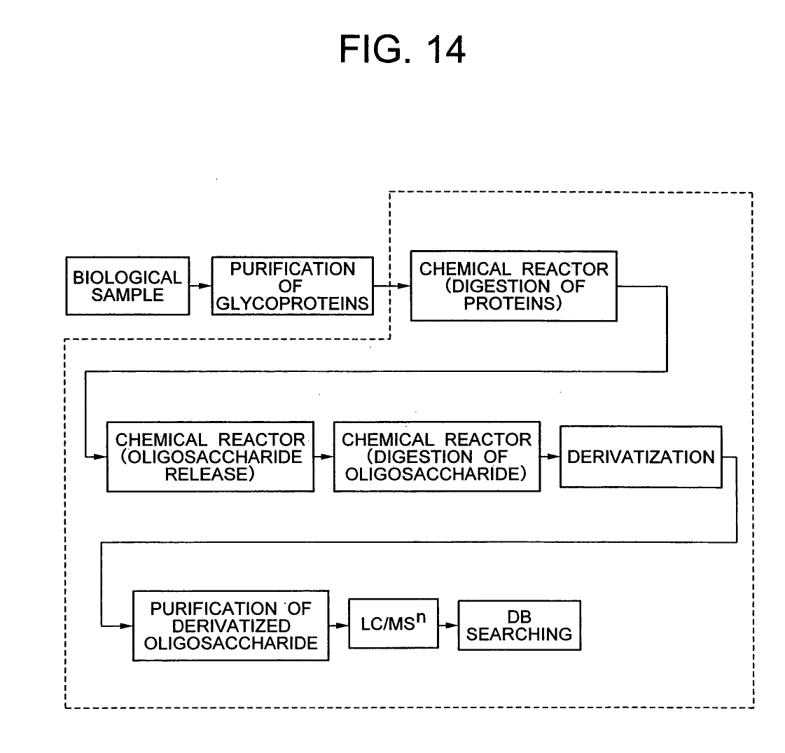

FIG. 14 shows a relation of a structural analysis (oligosaccharide sequence analysis) protocol of oligosaccharide using a mass spectrometer, and a diagram of analytical system in which the chemical reactor according to one example of the present invention is incorporated. In FIG. 14, the part enclosed with a dotted line is the analytical system in which the chemical reactor according to one example of the present invention is incorporated. In this case, three kinds of chemical reactions (protein digestion, oligosaccharide release, oligosaccharide digestion) are necessary, and three kinds of chemical reactors are used as shown in FIG. 14. These are different only in kind of enzyme immobilized, and structure, reaction time and temperature of the reaction part 1. Hitherto, about 2 days are required only for carrying out the three kinds of chemical reaction in batch-wise manner, while it can be shortened to about one hour by using the chemical reactor of the present invention. Here, it is also desired that the treating time in the chemical reactor is substantially the same as the actual reaction time, and a method of introducing the sample using the sample introduction parts as shown in FIG. 3 to FIG. 6 is effective.

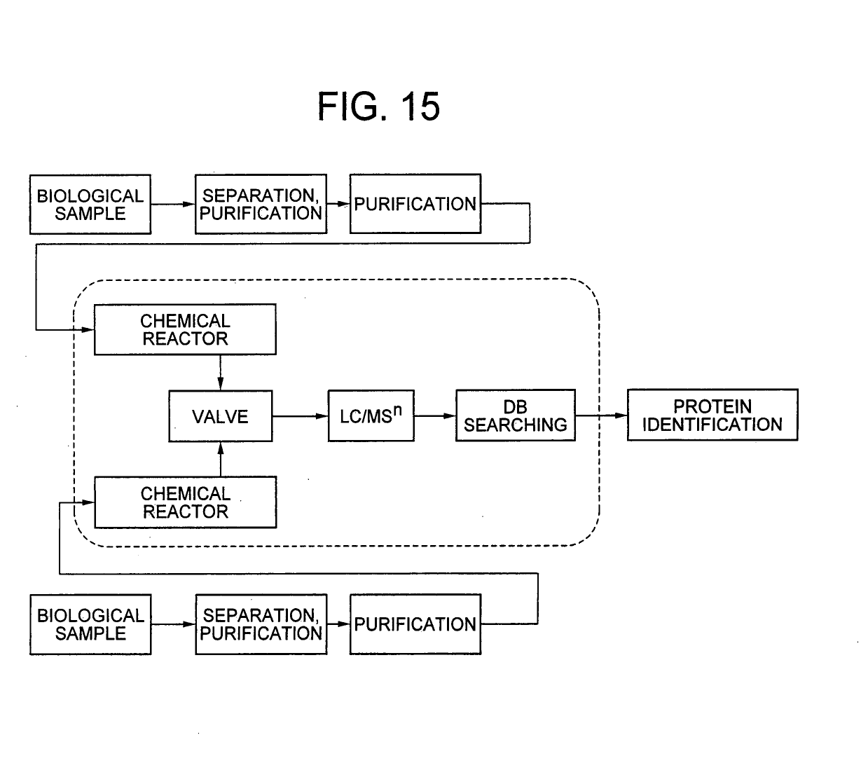

Furthermore, in case the liquid chromatograph completes in a short time in 1D-HPLC/MSⁿ system, a plurality of the chemical reactors can be operated in parallel as shown in FIG. 15. In such a high throughput analysis, the number of the chemical reactors can be optimized in view of the time required for separation in the liquid chromatograph.

It should be further understood by those skilled in the art that although the foregoing description has been made on embodiments of the invention, the invention is not limited thereto and various changes and modifications may be made without departing from the spirit of the invention and the scope of the appended claims.

1. A process of chemical reaction which comprises a step of introducing a first solution into a sample flow path including a reaction part containing a carrier, on the surface of which biomolecules are immobilized, a step of introducing into the sample flow path a second solution disposed being separated from the first liquid with a gas layer, a step of introducing a sample into the gas layer, and a step of transferring the first solution, the sample and the second solution so that the sample transfers relatively with the carrier.

2. A process of chemical reaction according to claim 1, wherein a first gas layer is present between the first solution and the sample and a second gas layer is present between the second solution and the sample.

3. A process of chemical reaction according to claim 2, wherein the volume of the first gas layer and the volume of the second gas layer are in the range of 0.1-2 μL, respectively.

4. A process of chemical reaction according to claim 1, wherein the volume of the sample introduced is not less than 0.1 μL and not more than 100 μL in the step of introducing the sample.

5. A process of chemical reaction according to claim 1, wherein the carrier comprises a plurality of fine particles and the reaction part is a capillary.

6. A process of chemical reaction according to claim 1, wherein the carrier is a structure provided in the reaction part and the reaction part is a capillary.

7. A chemical reactor which has a reaction part containing a plurality of fine particles, a first tube and a second tube connected with one end and another end of the reaction part, respectively, a sample introduction means which is connected with the first tube and introduces a sample, and a first pump and a second pump for controlling the transfer of the sample in the reaction part.

8. A chemical reactor according to claim 7, wherein the transfer of the sample comprises reciprocation.

9. A chemical reactor according to claim 7, wherein the sample introduction means has at least a first flow path and a second flow path, and the disposition of the first flow path and that of the second flow path into which the sample is introduced are changed over by rotation to introduce the sample introduced into the second flow path into the reaction part.

10. A chemical reactor according to claim 7, wherein the sample introduction means has a sample holding part, and the sample introduced into the sample holding part is forced out by a gas or liquid subsequently introduced into the sample holding means to introduce the sample into the reaction part.

11. A chemical reactor according to claim 7 which further has a thermal chamber, in which the sample introduction means and the reaction part are provided in the thermal chamber.

12. A chemical reactor according to claim 7 which further has a thermal chamber, in which the reaction part is provided in the thermal chamber.

13. A chemical reactor according to claim 7 which further has a temperature controller for controlling the temperature of the reaction part.

14. A chemical reactor which has a first flow path, a second flow path provided with a reaction part containing a plurality of fine particles, a member for changing over the disposition of the first flow path and that of the second flow path, a first tube connected with one end of the first flow path or the second flow path, a second tube connected with another end of the first flow path or the second flow path, a first pump connected with the first tube, and a second pump connected with the second tube.

15. An analytical system which has a chemical reactor provided with a reaction part containing a plurality of fine particles, a first tube and a second tube connected with one end and another end of the reaction part, respectively, a sample introduction means connected with the first tube and introducing the sample, and a first pump and a second pump for controlling the transfer of the sample in the reaction part and which further has a transport pipe for transporting the sample discharged from the chemical reactor, a liquid chromatograph part connected with the transport pipe, a mass spectrometer into which the sample separated in the liquid chromatograph part is introduced, and a means for obtaining an output of the mass spectrometer.

16. An analytical system according to claim 15 which has a plurality of the chemical reactors, in each of which enzyme is immobilized on the fine particles.
